(19) United States
(12) Patent Application Publication
(10) Pub. No.: US 2002/0161607 A1
(43) Pub. Date: Oct. 31, 2002

(54) Pharmaceutical drug sample tracking and control method

(76) Inventor: David C. Subich, Mansfield, OH (US)

Correspondence Address: Paul S. Rooy, 2620 S. Peninsula Dr, Dayton Beach, FL, 32118, US

(21) Appl. No.: 09/790,385

(22) Filed: Feb. 23, 2001

(51) Int. Cl. G06F 17/60; A61B 5/00

(52) U.S. Cl. 705/3; 600/300

(57) ABSTRACT

A pharmaceutical drug sample tracking and control method. Pharmaceutical drug manufacturers send pharmaceutical drug samples bearing pharmaceutical drug sample indicia to dispensing locations where an indicia reader is used to read the pharmaceutical drug sample indicia into a dispensing location database. As pharmaceutical drug samples are dispensed to patients, an indicia reader is used to read the pharmaceutical drug sample indicia on pharmaceutical drug samples thus dispensed into the dispensing location database. Inventory software tracks inventory levels by subtracting pharmaceutical drug samples dispensed to patients from the previous recorded inventories. Pharmaceutical drug manufacturers can access inventory levels at dispensing locations remotely. Practitioners enter patient information, practitioner information, adverse reaction information and patient outcomes in the dispensing location database. Interested parties can access this information. Pharmaceutical drug manufacturers may instruct an intermediary to determine patient test group lists, patient newsletter lists, and lists of patients to whom drug recall and/or advisory information should be sent, and may further instruct the intermediary to send appropriate invitations, newsletters, and information to such patients, thereby maintaining patient confidentiality.

### BACKGROUND OF THE INVENTION

1. Field of the Invention

This invention relates to pharmaceutical drug inventory systems, and in particular to a pharmaceutical drug sample tracking and control method.

2. Background of the Invention

It has become standard practice in the pharmaceutical drug industry for pharmaceutical drug manufacturers to provide no-cost pharmaceutical drug samples to health practitioners for distribution to their patients. The motivation for pharmaceutical drug manufacturers to make samples available free is the prospect that if the pharmaceutical drug sample achieves a desired result, the patient will continue to buy the drug from the pharmaceutical drug manufacturer in the future.

This type of pharmaceutical drug sample program is commonplace in such environments as medical offices, dentist offices, and veterinarian offices. A typical scenario may involve a patient being diagnosed, and an initial prescription being filled by means of drug samples given the patient by the diagnosing office. As the patient progresses on the pharmaceutical drug regimen, the patient is monitored to detect any adverse reactions to the drug, as well as to ascertain the patient outcome from undergoing the pharmaceutical drug regimen.

A major advantage to the sample approach is the way that this type of program dovetails with the monitoring of adverse reactions in patients. Whether or not a patient will experience adverse reaction(s) depends in part on any other pharmaceutical drugs the particular patient may be taking. Given the plethora of pharmaceutical drugs available, complete adverse reaction and contraindication data does not exist on every possible combination of pharmaceutical drugs a patient could conceivably take. Therefore, it would be desirable to provide a method to capture this type of information, so as to broaden the existing database of information regarding pharmaceutical drug adverse reactions and contraindications, especially with a combination of other pharmaceutical drugs.

One drawback with the current pharmaceutical drug sample programs is the difficulty of knowing when sample stocks at a given dispensing location require replenishment. It would be desirable for a pharmaceutical drug manufacturer to be able to check the levels of the pharmaceutical drug samples at various dispensing locations without having to actually visit the dispensing locations.

Another drawback with the current pharmaceutical drug sample programs is the inability of pharmaceutical drug manufacturers to quickly identify a pool of patients who would be eligible to participate in pharmaceutical drug testing programs. Typically such pharmaceutical drug testing subjects must conform with physical requirements, and sometimes they must also be taking a specific combination of other pharmaceutical drugs. Thus, it would be desirable to provide a pharmaceutical drug sample tracking and control method which would afford the capability of quickly and easily determining a pool of potential subjects eligible to participate in a given pharmaceutical drug study.

Additionally, it would be useful to provide a pharmaceutical drug sample tracking and control method which would readily yield lists of patients taking specific pharmaceutical drugs, or specific combinations of pharmaceutical drugs. In addition, it would be useful to provide a pharmaceutical drug sample tracking and control method which would be searchable in accordance with criteria specifying physical patient condition parameters. Such lists could be used as mailing lists for various publications, including newsletters, appropriate for such patient types.

From time to time it becomes necessary for pharmaceutical drug manufacturers to send out advisory information, or even recall information, regarding a specific pharmaceutical drug. In such event, it would be useful for a pharmaceutical drug manufacturer to be able to quickly generate a list of patients who are taking a specific pharmaceutical drug, in order to quickly communicate such information to them.

Finally, there is a trend amongst the states to require that drug samples be traceable from pharmaceutical drug manufacturer all the way to the final patient using the pharmaceutical drug sample. For example, the state of Ohio enacted a statute which recently went into effect requiring that records be maintained tracking pharmaceutical drug samples from the manufacturer to the patient using them. Increasingly, an effective pharmaceutical drug sample tracking and control method is becoming not only a convenience, but a matter mandated by law.

Existing Designs

One approach to the problem of tracking and controlling the dispensation of medical items was taught in McGrady et al., U.S. Pat. No. 5,912,818. This patent taught a Local Area Network (“LAN”) to which a medical dispenser, various terminals, and bar code readers were connected. Although this system provided for tracking inventory levels of various medical items by bar codes read into a computer, the ′818, method provided slots to dispense only approximately 24 samples. This limitation was significant in view of the fact that many dispensing locations stock 300 plus pharmaceutical drug samples. In addition, the ′818 patent taught no means of identifying prospective pharmaceutical drug test subjects, nor of generating reader mailing lists for ailment/medication specific recipients. Finally, the ′818 patent taught no means of generating mailing lists for drug recall information.

In the broader sphere, electronic medical record systems exist in software form. These software programs are basically digital versions of the traditional paper records predominant in most medical offices, and provide no capability of tracking and controlling pharmaceutical drug samples.

### SUMMARY OF THE INVENTION

Accordingly, it is an object of the present invention to provide a pharmaceutical drug sample tracking and control method which tracks all pharmaceutical drug samples at a given dispensing location. Design features allowing this object to be accomplished include at least one pharmaceutical dug manufacturer dispensing pharmaceutical drug samples to at least one dispensing location and an indicia reader at the dispensing location which reads pharmaceutical drug sample indicia into a dispensing location database upon receipt of the samples, and upon dispensing the samples. Advantages associated with the accomplishment of this object include the ability of a pharmaceutical drug manufacturer to quickly and easily ascertain the inventory levels of pharmaceutical drug samples at a given dispensing location, including the expiration date of same. Where the pharmaceutical drug manufacturer communicates with the dispensing location database by means of an internet, the pharmaceutical drug manufacturer would be able to perform the above functions from his own location, without having to physically visit the dispensing locations.

It is another object of the present invention to provide a pharmaceutical drug sample tracking and control method which associates patient information with pharmaceutical drug types in retrievable and searchable form in a database. Design features allowing this object to be accomplished include at least one dispensing location database containing patient information associated with the pharmaceutical drugs they are taking. Benefits associated with the accomplishment of this object include the ability for practitioners to track patient outcomes and/or adverse reactions to specific pharmaceutical drug regimes. Where the practitioner communicate with the dispensing location database by means of an internet, the practitioner would be able to perform the above functions from his own location, without having to physically visit the dispensing location where the dispensing location database is located.

It is still another object of this invention to provide a pharmaceutical drug sample tracking and control method which permits pharmaceutical drug manufacturers to search dispensing location databases through an intermediary for lists of patients which conform with defined criteria, such as pharmaceutical drugs being taken and patient physical characteristics. Design features enabling the accomplishment of this object include at least one dispensing location database containing patient information associated with the pharmaceutical drugs they are taking. Advantages associated with the realization of this object include the capability of identifying patient test group lists containing patients eligible to participate in pharmaceutical drug testing, and the capability of generating patient lists containing the names of patients who may be interested in receiving informational newsletters which contain advertising for pharmaceutical drugs.

It is still another object of this invention to provide a pharmaceutical drug sample tracking and control method which permits pharmaceutical drug manufacturers to search dispensing location databases through an intermediary for lists of patients by pharmaceutical drugs being taken. Design features enabling the accomplishment of this object include at least one dispensing location database containing patient information associated with the pharmaceutical drugs they are taking. Advantages associated with the realization of this object include the ability to identify patient lists containing names of patients to whom to send drug recall information and/or drug advisory information.

It is another object of the present invention to provide a pharmaceutical drug sample tracking and control method which aids in the development of nation-wide treatment protocols. Design features allowing this object to be accomplished include at least one dispensing location database connected to a central database by means of an internet. Benefits associated with the accomplishment of this object include enhanced effectiveness of pharmaceutical drug protocols, and the associated cost savings.

It is still another object of the present invention to provide a pharmaceutical drug sample tracking and control method which permits pharmaceutical drug manufacturers to contact patients who may be eligible to participate in pharmaceutical drug testing programs, or who may wish to receive pharmaceutical drug-specific newsletters, or who should receive pharmaceutical drug advisories and/or recall information, without making lists of such patients available to the pharmaceutical drug manufacturers. Design features allowing this object to be accomplished include at least one dispensing location database containing patient information associated with the pharmaceutical drugs they are taking, and at least one pharmaceutical drug manufacturer communicating with the dispensing location database(s) through an intermediary. A benefit associated with the accomplishment of this object is preservation of patient confidence.

It is yet another object of this invention to provide a pharmaceutical drug sample tracking and control method which uses existing hardware and equipment. Design features allowing this object to be achieved include the use of readily available components such as computers, bar code readers, and the internet. Benefits associated with reaching this objective include reduced cost, and hence increased availability.

### BRIEF DESCRIPTION OF THE DRAWINGS

The invention, together with the other objects, features, aspects and advantages thereof will be more clearly understood from the following in conjunction with the accompanying drawings.

Four sheets of drawings are provided. Sheet one contains FIG. 1. Sheet two contains FIG. 2. Sheet three contains FIG. 3. Sheet four contains FIG. 4.

FIG. 1 is a diagrammatic view of the instant pharmaceutical drug sample tracking and control method.

FIG. 2 is a diagrammatic view of that portion of the instant pharmaceutical drug sample tracking and control method wherein a pharmaceutical drug manufacturer sends pharmaceutical drug samples to a dispensing location, where an indicia reader reads pharmaceutical drug sample indicia into a dispensing location database.

FIG. 3 is a diagrammatic view of that portion of the instant pharmaceutical drug sample tracking and control method wherein pharmaceutical drug samples are dispensed to a patient, and the transaction is read into a dispensing location database by means of an indicia reader. In addition, a practitioner enters patient and practitioner information into the dispensing location database.

FIG. 4 is a diagrammatic view of that portion of the instant pharmaceutical drug sample tracking and control method wherein a pharmaceutical drug manufacturer sends a query into a central database and software through an intermediary, and a patient test group list or patient newsletter list is returned to the intermediary. The pharmaceutical drug manufacturer may then direct the intermediary to send drug recall information, drug advisory information, or pharmaceutical drug manufacturer's newsletters to patients, as appropriate. In this fashion patient confidentiality is preserved, because the pharmaceutical drug manufacturer never knows who the specific patient recipients are.

### DETAILED DESCRIPTION OF THE PREFERRED EMBODIMENT

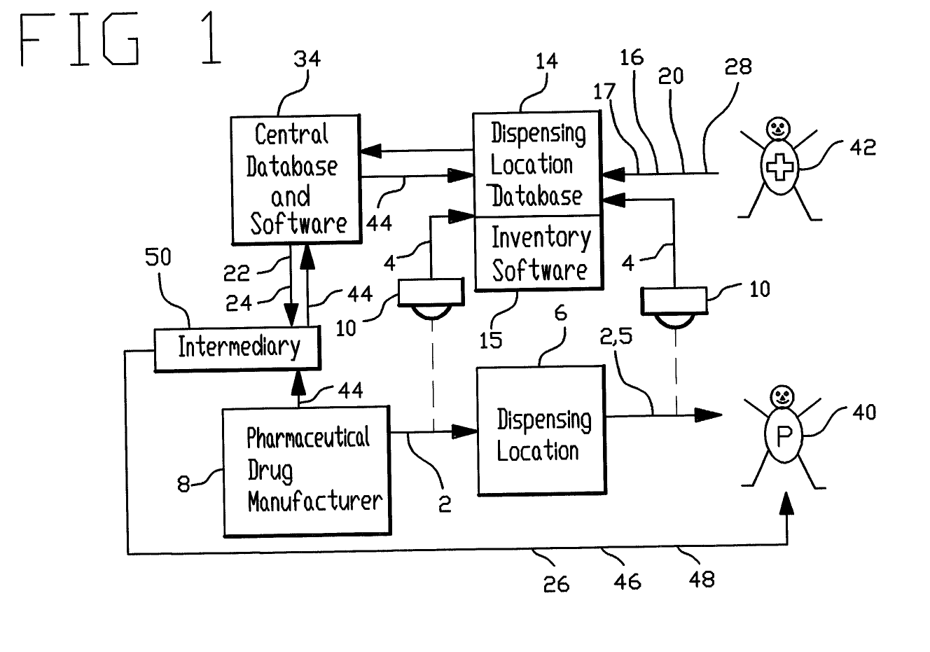

FIG. 1 is a diagrammatic view of the instant pharmaceutical drug sample tracking and control method. Pharmaceutical drug manufacturer 8 sends pharmaceutical drug samples 2 to dispensing location 6. Pharmaceutical drug samples 2 bear pharmaceutical drug sample indicia 4. Pharmaceutical drug sample indicia 4 may include drug type, lot number, expiration date, and dosage strength.

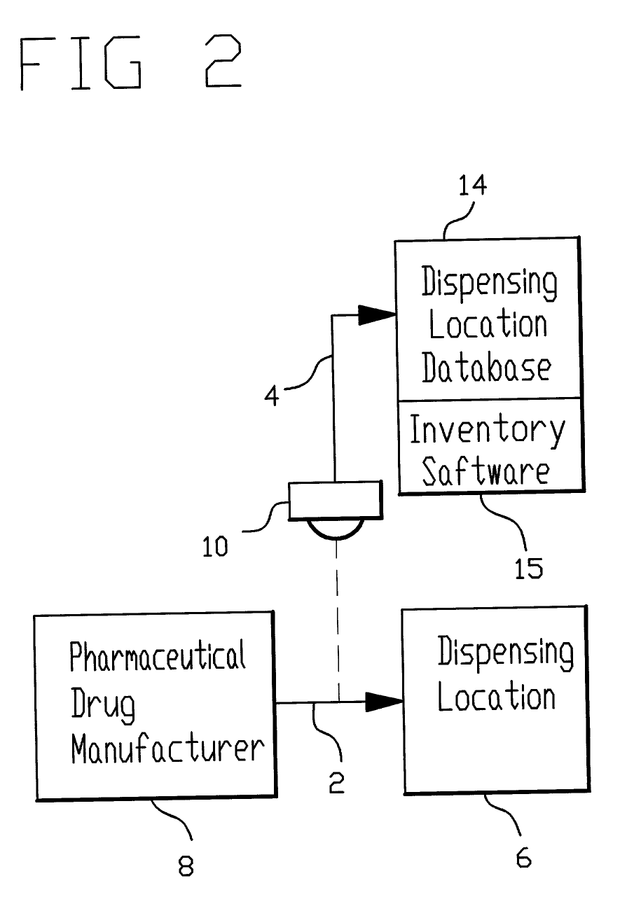

Referring now also to FIG. 2, as pharmaceutical drug samples 2 are received at dispensing location 6 their pharmaceutical drug sample indicia 4 is read by indicia reader 10, and the pharmaceutical drug sample indicia 4 is stored in dispensing location database 14 using inventory software 15.

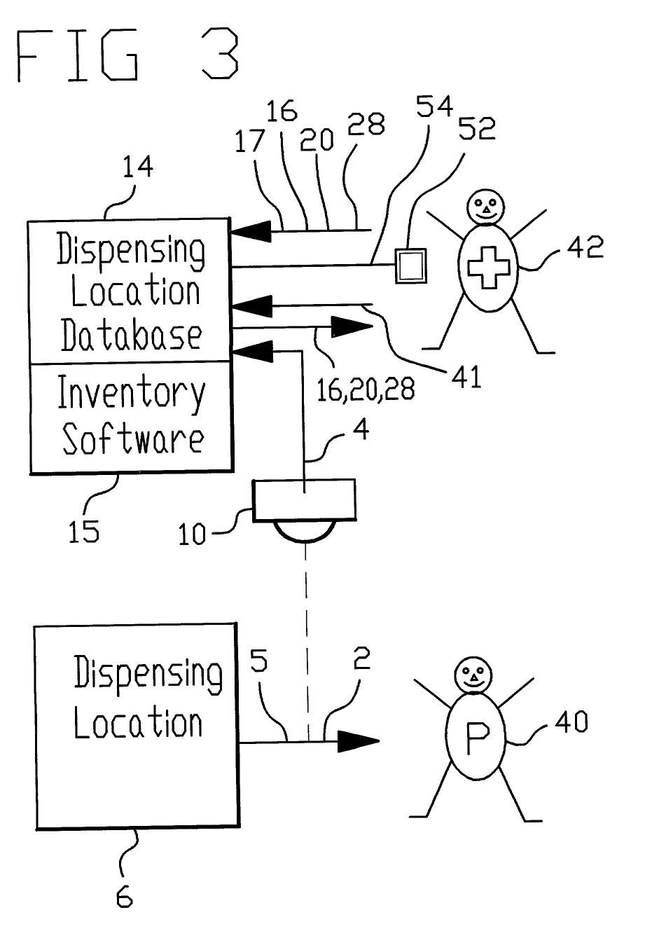

Also referring now to FIG. 3, pharmaceutical drug samples 2 are dispensed to patient 40. As pharmaceutical drug samples 2 are dispensed to patient 40, indicia reader 10 reads pharmaceutical drug sample indicia 4 into dispensing location database 14. Inventory software 15 subtracts the pharmaceutical drug samples 2 thus dispensed from the previous total inventory of same, and stock level of pharmaceutical drug samples 2 can be ascertained at any time by consulting inventory software 15. Where dispensing location database 14 and inventory software 15 are accessible via the internet, a remotely located pharmaceutical drug manufacturer 8 can simply go to the appropriate web address to ascertain what the inventory levels are for pharmaceutical drug samples 2 at any dispensing location 6 thus connected.

Practitioner 42 enters patient information 16, any adverse reaction information 20, and patient outcomes 28 into dispensing location database 14. In this fashion, an interested party may search dispensing location database 14 for data regarding the reactions of patients 40 to various pharmaceutical drug samples 2. This is illustrated in FIG. 3 by a practitioner 42 sending patient information query 41 to dispensing location database 14, and in return receiving patient information 16, adverse reaction information 20, and patient outcomes 28. In short, an interested party may access this information from any internet-accessible location.

For clarity, FIGS. 1-4 may depict only a single pharmaceutical drug manufacturer 8, dispensing location 6 with its associated dispensing location database 14 and inventory software 15, indicia readers 10, patients 40 and practitioners 42. In practice, many of each of these elements could exist. Central database and software 34 is connected to dispensing location databases 14 by means of the internet. Pharmaceutical drug manufacturers 8 may access their pharmaceutical drug sample 2 inventory levels at different dispensing locations 6 by means of their internet connections with the different dispensing location databases 14.

An additional feature of the instant pharmaceutical drug sample tracking and control method is depicted in FIG. 3. If certain patient 40 economic criteria are met, a given patient 40 may be eligible to receive voucher(s) 5 for pharmaceutical drugs. If such economic criteria are met, then the appropriate voucher(s) 5 may be quickly and easily printed out at dispensing location 6 as directed by a practitioner 42.

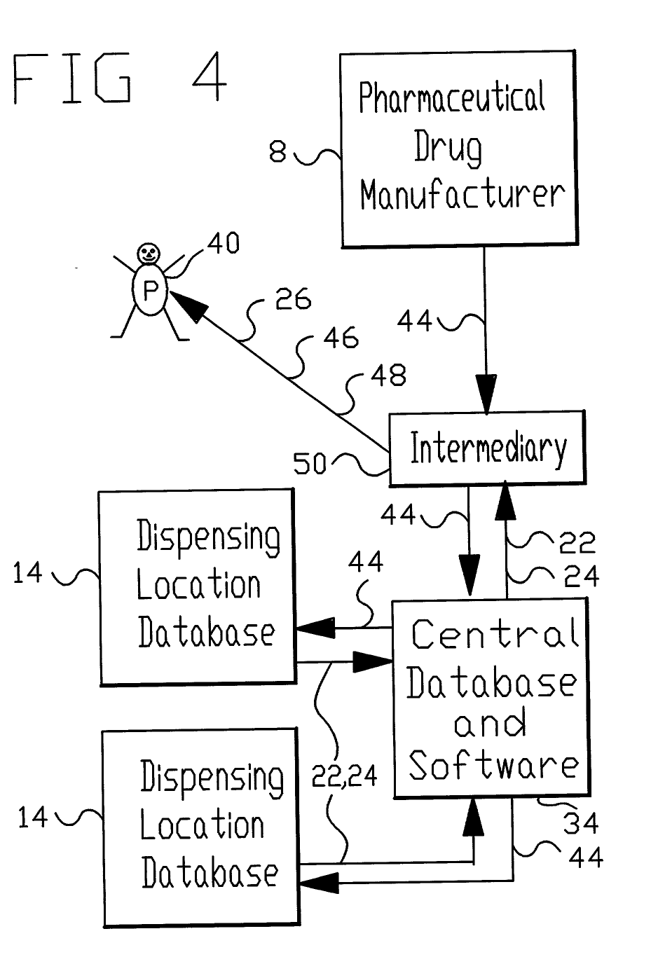

As may be observed in FIG. 4, a pharmaceutical drug manufacturer may send a query 44 into central database and software 34 through intermediary 50, and a patient test group list 22 or patient newsletter list 24 is returned to intermediary 50. In addition, based on this information sent to intermediary 50, pharmaceutical drug manufacturer 8 may instruct intermediary 50 to send drug recall information 46, drug advisory information 48, and/or pharmaceutical drug manufacturer's newsletters 26 to patients 40 and/or to affected practitioners 42. The transmission of such communications may be via e-mail, conventional mail, facsimile, or any other appropriate medium. In this fashion patient confidentiality is preserved, because the querying pharmaceutical drug manufacturer 8 never knows who the specific patient 40 recipients are.

Still another feature of the instant pharmaceutical drug sample tracking and control method is the ability for practitioner 42 prescription habit tracking to be obtained from dispensing location database 14 using inventory software 15. Thus, each time a practitioner 42 dispenses pharmaceutical drug samples 2, such practitioner 42 enters practitioner information 17 into his dispensing location database 14. Inventory software 15 keeps track of the prescription drug habits of each practitioner 42 using the system. Over time, the prescription history of each practitioner 42 is established, and prescription tracking accessed for such practitioner 42.

Yet another feature of the instant pharmaceutical drug sample tracking and control method is the ability for practitioner 42 to sign for pharmaceutical drug samples 2 using digital signature pad 52 as depicted in FIG. 3. It is standard practice in the industry to require a practitioner 42 to sign for pharmaceutical drug samples 2 before receiving same. The instant method permits the signature of practitioner 42 to be digitally transmitted to pharmaceutical drug manufacturers 8 via dispensing location database 14 and central database and software 34, and/or via the internet. Upon receipt of digital signature 54, the pharmaceutical drug manufacturer 8 receiving digital signature 54 sends pharmaceutical drug samples 2 via mail, courier, etc. In addition, it is contemplated that software implementing the instant pharmaceutical drug sample tracking and control method would include the capability of digital video conferencing using existing digital camera technology, whereby pharmaceutical drug manufacturer's representatives would be able to video conference, face-to-face, with practitioners 42 at dispensing locations 6, thus avoiding the time and expense of personal visits.

The pharmaceutical drug sample tracking and control method comprises the following steps:

A. Receiving pharmaceutical drug samples identified by pharmaceutical drug sample indicia at one or more dispensing locations from one or more pharmaceutical drug manufacturers, said pharmaceutical drug sample indicia comprising information including pharmaceutical drug type, lot number, expiration date, and dosage strength;

B. Using one or more indicia readers to read the pharmaceutical drug sample indicia on each pharmaceutical drug sample received, and storing the pharmaceutical drug sample indicia information thus read in at least one dispensing location database on a dispensing location computer;

C. Dispensing pharmaceutical drug samples to patients, using an indicia reader to read the pharmaceutical drug sample indicia on each pharmaceutical drug sample dispensed, and storing the pharmaceutical drug sample indicia information thus read in at least one dispensing location database;

D. When dispensing pharmaceutical drug samples to patients, entering patient information into said dispensing location database, said patient information including patient allergies, other medications and/or nutriceuticals that the patient is taking, possible drug interactions, and the daily dosage and duration of the sample prescription;

E. In the event a patient experiences an adverse reaction to a pharmaceutical drug sample, entering the adverse reaction information into an appropriate dispensing location database, and also forwarding same to appropriate sources such as the Food and Drug Administration, the affected pharmaceutical drug manufacturer, etc.;

F. As the sample pharmaceutical regimen progresses, entering patient outcomes attributable to the sample pharmaceutical regimen into the appropriate dispensing location database as the outcomes are observed;

G. Interested parties sending a patient information query to an appropriate dispensing location database, and thereby determining patient sample dispensation histories and patient information;

H. Pharmaceutical drug manufacturers accessing said dispensing location database and determining inventory levels of pharmaceutical drug samples at one or more dispensing locations, including the expiration dates of samples, using inventory software;

I. Pharmaceutical drug manufacturers searching the information residing in at least one dispensing location database through an intermediary to determine suitable patient test groups lists for pharmaceutical drug testing; and

J. Pharmaceutical drug manufacturers searching patient information residing in at least one dispensing location database through an intermediary to determine suitable patient newsletter lists to which to send pharmaceutical drug manufacturer's newsletters.

The pharmaceutical drug sample tracking and control method may comprise one or more of the following additional steps:

K. Using one or more bar code readers to read the pharmaceutical drug sample indicia on each pharmaceutical drug sample received, and storing the pharmaceutical drug sample indicia information thus read in at least one dispensing location database.

L. Dispensing pharmaceutical drug samples to patients, using said bar code reader to read the pharmaceutical drug sample indicia on each pharmaceutical drug sample dispensed, and storing the pharmaceutical drug sample indicia information thus read in at least one dispensing location database.

M. Using palm computers to enter said patient information, patient outcomes and adverse reactions into said dispensing location database.

N. Providing a central database connected to each dispensing location database through at least one firewall by means of an internet.

O. Sending a query for a list of prospective pharmaceutical drug test subjects which conform to specified physical and/or other drug(s) being taken criteria from at least one said pharmaceutical drug manufacturer to said central database through an intermediary, and thence to at least one said dispensing location database, sending a resulting patient test group list back to said intermediary, and said intermediary sending out invitations to participate in a pharmaceutical drug testing program to patients listed on said patient test group list.

P. Sending a query for a patient newsletter list for ailment/medication specific recipients from at least one said pharmaceutical drug manufacturer to said central database through an intermediary, and thence to at least one said dispensing location database, sending search results back to said intermediary, and said intermediary sending newsletters to patients listed on a resulting patient newsletter list.

Q. Sending a query for a mailing list for drug recall or advisory information from at least one said pharmaceutical drug manufacturer to said central database through an intermediary, and thence to at least one said dispensing location database, sending search results back to said intermediary, and said intermediary sending said drug recall information and/or drug advisory information to patients listed in said search results and/or to affected practitioners.

R. Pharmaceutical drug manufacturers accessing the dispensing location database via an internet to determine inventory levels of pharmaceutical drug samples at one or more dispensing locations, including the expiration dates of samples.

S. When dispensing pharmaceutical drug samples to patients, entering practitioner information into said dispensing location database, said practitioner information including the identification of the practitioner, the date, the patient, the patient condition, and the prescription, whereby practitioner prescription habit tracking may be provided.

T. A practitioner using a digital signature pad to send a digital signature to a pharmaceutical drug manufacturer by means of one said dispensing location database and said central database and software, or via an internet, and said pharmaceutical drug manufacturer sending a pharmaceutical drug sample in return.

U. A practitioner communicating with a pharmaceutical drug manufacturer by means of digital cameras at said dispensing location database at said pharmaceutical drug manufacturer, through said central database and software, via an internet.

V. Said dispensing location issuing pharmaceutical drug vouchers to eligible patients by quickly and easily printing out said vouchers at said dispensing location as directed by a practitioner.

While a preferred embodiment of the invention has been illustrated herein, it is to be understood that changes and variations may be made by those skilled in the art without departing from the spirit of the appending claims.

DRAWING ITEM INDEX

| | |
|---|---|
| 2 | pharmaceutical drug samples |
| 4 | pharmaceutical drug sample indicia |
| 5 | voucher |
| 6 | dispensing location |
| 8 | pharmaceutical drug manufacturer |
| 10 | indicia reader |
| 14 | dispensing location database |
| 15 | inventory software |
| 16 | patient information |
| 17 | practitioner information |
| 20 | adverse reaction information |
| 22 | patient test group list |
| 24 | patient newsletter list |
| 26 | pharmaceutical drug manufacturer's newsletter |
| 28 | patient outcome |
| 34 | central database and software |
| 40 | patient |
| 41 | patient information query |
| 42 | practitioner |
| 44 | query |
| 46 | drug recall information |
| 48 | drug advisory information |
| 50 | intermediary |
| 52 | digital signature pad |
| 54 | digital signature |

I claim:

1. A pharmaceutical drug sample tracking and control method comprising the steps of:
A. Receiving pharmaceutical drug samples identified by pharmaceutical drug sample indicia at one or more dispensing locations from one or more pharmaceutical drug manufacturers, said pharmaceutical drug sample indicia comprising information including pharmaceutical drug type, lot number, expiration date, and dosage strength;
B. Using one or more indicia readers to read said pharmaceutical drug sample indicia on each said pharmaceutical drug sample received, and storing said pharmaceutical drug sample indicia information thus read in at least one dispensing location database;
C. Dispensing said pharmaceutical drug samples to patients, using an indicia reader to read said pharmaceutical drug sample indicia on each said pharmaceutical drug sample dispensed, and storing the pharmaceutical drug sample indicia information thus read in at least one dispensing location database;
D. When dispensing pharmaceutical drug samples to patients, entering patient information into said dispensing location database, said patient information including patient allergies, other medications and/or nutriceuticals that said patient is taking, possible drug interactions, and daily dosage and duration of the sample prescription;
E. If said patient experiences an adverse reaction to said pharmaceutical drug sample, entering adverse reaction information into an appropriate said dispensing location database, and also forwarding said adverse reaction information to appropriate sources such as a Food and Drug Administration, and an affected pharmaceutical drug manufacturer;
F. As a sample pharmaceutical regimen progresses, entering patient outcomes attributable to said sample pharmaceutical regimen into an appropriate dispensing location database as said outcomes are observed; and
G. Pharmaceutical drug manufacturers accessing said dispensing location database and determining inventory levels of said pharmaceutical drug samples at said one or more dispensing locations, including expiration dates of said pharmaceutical drug samples by means of inventory software.

2. The pharmaceutical drug sample tracking and control method of claim 1 further comprising the step of interested parties sending a patient information query to an appropriate dispensing location database, and thereby determining patient sample dispensation histories and patient information.

3. The pharmaceutical drug sample tracking and control method of claim 1 further comprising the following steps:
H. Sending a query for a list of prospective pharmaceutical drug test subjects which conform to specified physical and/or other drug(s) being taken criteria from at least one said pharmaceutical drug manufacturer to a central database through an intermediary, and thence to at least one said dispensing location database,
I. Sending a resulting patient test group list back to said intermediary; and
J. Said intermediary sending out invitations to participate in a pharmaceutical drug testing program to patients listed on said patient test group list.

4. The pharmaceutical drug sample tracking and control method of claim 1 further comprising the following steps:
H. Sending a query for a patient newsletter list for ailment/medication specific recipients from at least one said pharmaceutical drug manufacturer to a central database through an intermediary, and thence to at least one said dispensing location database,
I. Said intermediary receiving a patient newsletter list; and
J. Said intermediary sending out newsletters to patients listed on said patient newsletter list.

5. The pharmaceutical drug sample tracking and control method of claim 1 further comprising the following steps:
H. Sending a query for a mailing list for drug recall or drug advisory information from at least one said pharmaceutical drug manufacturer to a central database through an intermediary, and thence to at least one said dispensing location database;
I. Sending search results back to said intermediary; and
J. Said intermediary sending said drug recall information and/or drug advisory information to patients listed in said search results, and/or to affected practitioners.

6. The pharmaceutical drug sample tracking and control method of claim 1 further comprising the steps of using one or more bar code readers to read said pharmaceutical drug sample indicia on each said pharmaceutical drug sample received, and storing said pharmaceutical drug sample indicia information thus read in at least one said dispensing location database.

7. The pharmaceutical drug sample tracking and control method of claim 1 further comprising the steps of dispensing pharmaceutical drug samples to patients, using said bar code reader to read said pharmaceutical drug sample indicia on each pharmaceutical drug sample dispensed, and storing said pharmaceutical drug sample indicia information thus read in at least one said dispensing location database.

8. The pharmaceutical drug sample tracking and control method of claim 1 further comprising the step of using at least one palm computer to enter said patient information, patient outcomes and adverse reactions into said dispensing location database.

9. The pharmaceutical drug sample tracking and control method of claim 1 further comprising the step of providing a central database connected to each said dispensing location database through at least one firewall by means of an internet.

10. The pharmaceutical drug sample tracking and control method of claim 1 further comprising the step of pharmaceutical drug manufacturers accessing one or more said dispensing location databases via an internet to determine inventory levels of said pharmaceutical drug samples at said one or more dispensing locations, including expiration dates of said pharmaceutical drug samples, said pharmaceutical drug sample inventory levels being determined by inventory software which tracks said inventory levels by subtracting pharmaceutical drug samples dispensed to patients from previous recorded inventories.

11. The pharmaceutical drug sample tracking and control method of claim 1 further comprising the step of entering practitioner information into said dispensing location database when dispensing pharmaceutical drug samples to patients, said practitioner information including an identification of said practitioner, a date, a patient, condition of said patient, and a prescription, whereby practitioner prescription habit tracking may be provided.

12. The pharmaceutical drug sample tracking and control method of claim 1 further comprising the step of a practitioner using a digital signature pad to send a digital signature to a pharmaceutical drug manufacturer by means of one said dispensing location database and a central database and software, or via an internet, and said pharmaceutical drug manufacturer sending a said pharmaceutical drug sample in return.

13. The pharmaceutical drug sample tracking and control method of claim 1 further comprising the step of a practitioner communicating with a pharmaceutical drug manufacturer by means of digital cameras at said dispensing location database and at said pharmaceutical drug manufacturer, through said central database and software, via an internet.

14. The pharmaceutical drug sample tracking and control method of claim 1 further comprising the step of said dispensing location issuing pharmaceutical drug vouchers to eligible patients by quickly and easily printing out said vouchers at said dispensing location as directed by a practitioner.
